(19) United States
(12) Patent Application Publication
(10) Pub. No.: US 2011/0044969 A1
(43) Pub. Date: Feb. 24, 2011

(54) Antimicrobial Agents for the Treatment of Campylobacter Species in the Crop of a Bird

(75) Inventors: Knut Rudi, Osloveien (NO); Magne Kaldhusdal, Oslo (NO); John Nordal, Oslo (NO)

Correspondence Address: CANTOR COLBURN LLP, 20 Church Street, 22nd Floor, Hartford, CT, 06103, US

(73) Assignee: NOFIMA MAT AS, As (NO)

(21) Appl. No.: 12/866,098

(22) PCT Filed: Feb. 9, 2009
(86) PCT No.: PCT/GB09/00361

§ 371 Date: Nov. 2, 2010

(30) Foreign Application Priority Data
Feb. 7, 2008 (GB) 0802295.6

(51) Int. Cl. A61K 38/47; A61K 31/192; A61K 31/19; A61K 31/11; A61K 31/6615; A61K 36/45; A61K 36/8962; A61K 38/48; A61K 38/00; A61P 1/12

(52) U.S. Cl. 424/94.61; 514/568; 514/557; 514/699; 514/77; 424/732; 424/754; 424/94.63; 514/2.4

(57) ABSTRACT

The invention provides an antimicrobial agent for use in the treatment of a Campylobacter species which has colonised the crop of a bird, wherein the bird is exposed to said antimicrobial agent one or more times and products for carrying out the same.

The present invention relates to the care of birds, particularly to the care of chickens intended to enter the human food chain.

One of the most prominent emerging pathogen in the food chain is Campylobacter jejuni. C. jejuni is one of the leading causes of diarrhoeal disease and food-borne gastroenteritis in humans in the developed world. This bacterium is zoonotic and poultry is an important source for transmission to humans. C. jejuni is able to colonise the gastrointestinal (GI) tract of chickens and the principal site of colonisation is the lower GI tract, especially the caecum (Beery et al. 1988 Applied and Environmental Microbiology 54: 2365-2370). Despite major intervention efforts targeting the lower GI tract, no really successful approaches have been developed.

The upper part of the GI tract has not been considered as a potential reservoir for C. jejuni colonisation. Campylobacter has been detected in the crop, however it has only been considered as a transit of environmental contamination. Berrang et al. 2000 Poult Sci 79: 286-290 have tested for Campylobacter and other bacteria in the GI tract and on the feathers and skin of broilers, they reported the presence of bacteria in the crop but at lower levels than in the other GI regions tested (ceca and colon). These data would therefore not suggest the crop as a particular target for any attempts to reduce bacterial infection.

The present inventors have surprisingly found C. jejuni to colonise the crop mucosa, and shown that an antimicrobial treatment completely prevented the colonisation of C. jejuni in both crop and caecum. No previous studies have utilised the potential pathogen barrier effect of the crop. The present inventors have demonstrated modification of the crop environment as a surprising means for pathogen reduction in the bird.

Thus, the present invention provides an antimicrobial agent for use in the treatment of a Campylobacter species present in the crop of a bird, wherein the bird is exposed to said antimicrobial agent one or more times, preferably at least one of those exposures taking place during the early part of the bird's growth phase.

The esophagus of certain birds, particularly poultry, forms a pouch called a crop. From the crop food passes into the true stomach (proventriculus) and then to the gizzard and the rest of the GI tract. The present invention is applicable to any bird which has a crop and this includes poultry such as chicken, turkey, duck, geese, pheasant, partridge, guinea fowl and quail as well as other birds which possess a crop such as pigeon or dove. Although these latter two species are less frequently farmed for meat or eggs they may be reared by farmers or breeders. Galliform birds with well developed crops are particularly suitable and include the preferred target birds of chicken, turkey, guinea fowl, pheasant, partridge and quail. The methods and uses of the present invention are applicable to all farmed birds and birds reared with human intervention, this includes so called ‘battery farming’ as well as ‘free range’ methods. The invention is of utility whether the birds are reared for their eggs or meat but because one key aim is to reduce the entry of Campylobacter into the human food chain, the methods and uses of the invention are especially suitable for birds reared for their meat, such as broiler chickens.

The purpose of the present invention is to treat species of Campylobacter, particularly C. jejuni but also other species such as C. coli which are present in the crop. As described in the Examples, the present inventors have shown for the first time that Campylobacter colonise the crop in addition to being merely a contaminant of recently ingested crop contents. This new understanding makes the crop a surprising new target for antimicrobial treatments. Thus the uses and methods of the present invention are intended to target the crop in particular. While the treatments may also result in an antibacterial effect elsewhere in the GI tract, and this may offer additional benefits, the aim is to reduce or eliminate Campylobacter colonisation in the crop.

Thus in a preferred embodiment the present invention provides an antimicrobial agent for use in the treatment of a Campylobacter species which has colonised the crop of a bird, wherein the bird is exposed to said antimicrobial agent one or more times, preferably at least one of those exposures taking place during the early part of the bird's growth phase. A colonising population can be found in the mucosa of the crop whereas a contaminating population exists predominantly in the lumen of the crop. Therefore the antimicrobial agent or the composition in which it is administered can be adapted for targeting to the crop of a bird, preferably the mucosa. Thus preferred uses, treatments or exposures will be designed or selected to target the mucosa. Thus suitable compositions containing an antimicrobial agent may include those which adhere to the mucosa and suitable antimicrobial agents may be those which are transportable into the cells associated with the mucosa. Mucoadhesive compounds are discussed in Harding et al. 2003, Biochemical Society Transactions 31(5): 1036-1041.

The skilled man will be aware of techniques which may be employed to target the crop. Such techniques will preferably ensure that at least 30% more, preferably at least 5-0%, more preferably at least 70%, (e.g. 85%, 90% or 95%) of the antimicrobial activity of the administered antimicrobial agent will be localised to the crop as opposed to the remainder of the bird or, viewed more specifically, the remainder of the bird's digestive system.

The antimicrobial agents are typically administered with or as part of the birds' normal feed and/or drinking water. This provides a suitable way simply to target the crop. During feed uptake, food first accumulates in the gizzard, when the gizzard is full, further feed uptake induces accumulation of feed in the crop. In-feed antimicrobials can take advantage of this natural storage mechanism. In order to increase the amount of food consumed by a bird in one feeding phase, and therefore increase the volume and duration of feed in the crop, it is preferable to have a feeding regimen in which the birds do not have constant access to food.

Preferably the birds are denied access to feed for a time period sufficient to stimulate their appetite to a degree such that when feed is again made available, their consumption will be such as to cause accumulation of feed in the crop. This will increase the exposure of the in-feed antimicrobial agent to the crop environment, in particular exposure to the mucosa of the crop which the bacteria typically colonise. The length of time required to stimulate the appetite to this extent will vary from species to species and depend on the overall feeding regimen, typical time periods will be 2-8 hours, e.g. 3 to 5 hours, preferably around 4 hours. Thus, preferably, the bird is exposed to an in-feed antimicrobial agent after a period of feed withdrawal.

The above describes how the antimicrobial agent may be targeted to the crop by virtue of a method designed to retain the agent in the crop. As well or instead of retention, targeting may be achieved by activating or releasing the antimicrobial agent in the crop. An antimicrobial containing controlled release formulation may be prepared, again typically for inclusion in the feed, which results in selective release of the antimicrobial agent in the crop. Release may be based on the specific environment of the crop, whether that be pH or enzymatic etc.

In particular, the crop has a lower pH than more anterior parts of the GI tract and this can be utilised to cause release of the agent from a controlled release formulation which is sensitive to degradation at the appropriate pH.

Alternatively the release rate controlling agent could simply be a substance that allows the antimicrobial to be released in the time window corresponding to the time in which it takes feed to pass through the crop.

The antimicrobial agent will be active against Campylobacter species, particularly against C. jejuni. Suitable agents are discussed below and others are described in the literature and known to the skilled man and continue to be developed.

One particularly effective class of antimicrobial agents are weak-organic acids and their salts, for instance sorbates. Their bactericidal effect is dependent on low pH for optimal inhibitory activity and so will result in an effective targeting of antimicrobial activity to the crop. It may also be desirable to administer an enhancer as well as the primary antimicrobial agent to adjust the pH to optimise the antimicrobial agent's activity. The enhancer would typically be a proton donor, an acid such as formic acid, which lowers the pH within the feed or the crop and enhances the antimicrobial activity of an agent such as sorbate. The enhancer may preferably be present in the feed with the antimicrobial agent or in the drinking water.

It will usually be desirable, particularly when the antimicrobial agent is an acid or salt thereof, to ensure the bird has access to sufficient drinking water when feeding and/or moistened food so that the crop contents are moist, this is intended to encourage release of an active form of the antimicrobial agent.

Further enhancers or activators intended to cause release or activation of the antimicrobial agent from its formulation or generate its active form are enzymes. Enzymes may be present in the feed or drinking water.

Campylobacter is a known pathogen and various agents are known to have bactericidal or bacteristatic activity against it. For the present invention, bactericidal activity is preferred. Organic acids and their salts are a particularly preferred antimicrobial agent for use according to the invention, e.g. benzoic, formic or sorbic acid and their salts, e.g. K, Mg or Ca, especially potassium sorbate. Benzoic and sorbic acid and their salts are preferred, sorbic acid or its salts being most preferred. Further agents which have been reported to have anti-Campylobacter activity are benzaldehydes and azoles.

Inorganic antimicrobial agents are also suitable, such as trisodium phosphate.

The antimicrobial agent is preferably added to the birds' feed and natural plants, including berries, and extracts therefrom are particularly compatible with this mode of administration. These include cowberry, rowanberry, blueberry and leek and extracts therefrom. Where the antimicrobial agent is supplied as a berry or some other plant part it is usually desirable for an activator/enhancer to be co-administered, such an activator is typically a degrative enzyme (for instance, a pectinase, a cellulase, a protease, etc.) which can release the antimicrobial agent from, e.g., the berry. An enzymatic or other activator, for instance a pectinase etc., is preferably included in the drinking water which the birds receive to, inter alfa, promote release of the antimicrobial agent in the crop.

A further class of antimicrobial agent which is suitable for use with the present invention are antimicrobial peptides. Again these peptides, which may be membrane acting and have a cytolytic effect on the bacteria or have membrane bound or intra-cellular protein or other targets, are conveniently included in the bird feed. An activator would typically be a peptidase to cleave and thus release an active peptide, again the enzyme would preferably be included in the drinking water to, inter alia, promote release of the antimicrobial peptide in the crop.

One or more antimicrobial agents may be used in the treatments of the present invention.

As discussed above, exposure is typically achieved through adding or combining the antimicrobial agent, optionally in a formulation which also contains one or more carriers, diluents or excipients which may assist in e.g. the transport, storage or bioavailability of the antimicrobial agent, to or with the bird's feed or drinking water. However exposure may be achieved separately from the dietary regimen.

Feed formulations comprising an antimicrobial agent will typically comprise 0.01 to 5% by weight of the antimicrobial agent, more usually 0.025 to 1% of the antimicrobial agent. For a salt of an organic acid, for instance potassium sorbate, this range is suitable, a range of 0.05 to 0.5% being particularly preferred. An acid enhancer, for instance formic acid, will typically be present at 0.1 to 5%, preferably 0.5 to 3% by weight more preferably 1 to 2% e.g. around 1.5%.

The optimum proportion of antimicrobial agent and/or enhancer may be influenced by the identity of these agents and the conflict, if any, between obtaining a sufficiently effective treatment of Campylobacter colonisation and any growth depressing effects of the agents being used. It is routine for the skilled man to identify these optimum proportions.

In one embodiment potassium sorbate and formic acid are used as in-feed additives at between 0.1% and 0.5% and between 1% to 2% respectively.

The present invention is based on the new understanding that Campylobacter colonise the crop and therefore the crop is a target site for antimicrobial treatments. Although Beery et al. and Berrang et al., supra, found Campylobacter in the crop, they concluded that the bacteria primarily colonized the lower GI tract.

As well as colonization, studies have also been performed on the contamination of the carcass of broiler chickens around the time of slaughter. A standard management practice in commercial broiler production is the removal of feed some hours prior to processing, the purpose of which is to enhance the clearance of the GI tract. This withdrawal may last from 2-10 hours. However, as reported by Byrd et al. 1998 Avian Diseases 42: 802-806; feed withdrawal can increase the contamination by Campylobacter of the crop. It is postulated that methodologies aimed at reducing Campylobacter contamination of the crop at the end of the bird's life might be important for reducing carcass contamination. Byrd et al. teach that the crop is a site of secondary contamination from the caecum, most likely because chickens eat their own faeces upon feed withdraw. Thus the most plausible action based on the comments in Byrd et al. would be to prevent the chickens eating their own faeces or reduce the Campylobacter load in the caecum. In contrast, because the present inventors have identified the crop as a site of colonisation by Campylobacter at a relatively early stage in the life cycle of the chicken, the crop is now identified as a target site in its own right and not only after secondary contamination caused by feed withdrawal.

Thus, in a preferred embodiment, the birds are exposed to an antimicrobial agent in the early part of the bird's growth phase. ‘Early’ is used to differentiate from the phase shortly before, during or after the feed withdrawal before the birds are slaughtered, or where there is no feed withdrawal, shortly before slaughter. The life-span of the birds will vary depending on the species and the intensity of rearing; chickens bred for meat will usually be slaughtered between 28 and 45 days, although larger broilers reared under a free range approach may be slaughtered after up to 80 days. Thus the ‘early part’ will typically be all but the last 10, e.g. all but the last 5 days of the chicken's life. Alternately viewed, the ‘early part’ will typically be the first 20, at least the first 15 days of the chicken's life.

Suitable treatment regimen may involve exposure of the birds to an antimicrobial agent on a daily (or every 2 or 3 days) basis. Typically this regular exposure, e.g. though their feed or drinking water, will begin around day 7-14 of the bird's life, most suitably around day 10 however, earlier exposure (e.g. from hatching) is also contemplated. Suitable treatment regimens will involve multiple exposures depending on the time to slaughter of the birds, e.g. 2-40, more typically 5-25, e.g. 8-20.

As discussed above, in one embodiment of the invention the exposure may be after a period of feed-withdrawal, therefore the birds would have ‘meal times’ rather than continuous access to food during their growth period. This approach has independently been proposed to have benefits in terms of welfare as it promotes exercise.

Although at least one of the exposures is preferably in the early phase, the treatment regimen may involve exposures throughout the growth phase or even the whole life of the bird.

As an alternative to early stage exposure, the bird may receive a plurality of exposures in the period before and leading up to slaughter, e.g. 2-10, preferably 3 or 4 or more exposures in the 10 days, e.g. the last 7 days, prior to slaughter. Preferably the exposures will be daily.

Reference herein to ‘treatment’ of a Campylobacter species includes a reduction in detectable numbers of the bacteria. Total elimination, while desirable, is not required for a useful treatment. Treatment may involve a bactericidal or bacteristatic effect and thus a ‘reduction’ in the Campylobacter population may only be relative to what would have developed without treatment and/or only seen over longer periods as the inhibition of growth and multiplication manifests itself. The treatment will preferably be prophylactic, serving to restrict or prevent colonisation before it has occurred or developed significantly. Thus again, a reduction in bacterial numbers is as compared to an expected, untreated, progression.

In a further aspect the present invention provides a method of treating a Campylobacter species present in the crop of a bird, wherein the bird is exposed to an antimicrobial agent one or more times, preferably at least one of those exposures taking place during the early part of the bird's growth phase. The above described preferred features of the veterinary use of the invention also apply mutatis mutandis to this aspect of the invention.

In a further aspect the present invention provides a product containing (a) an antimicrobial agent, and (b) an activator and/or enhancer of said antimicrobial agent, as a combined preparation for separate, simultaneous or sequential use in the treatment of a Campylobacter species in the crop of a bird, wherein the bird is exposed to said antimicrobial agent one or more times, preferably at least one of those exposures taking place during the early part of the bird's growth phase. The antimicrobial agent and activator thereof are as described above, the activator typically being a pH moderator, e.g. an acid or an enzyme which serves to release the active antimicrobial agent. The above described preferred features of the veterinary use of the invention also apply mutatis mutandis to this aspect of the invention.

The invention will now be described with reference to the following non-limiting Examples and the FIGURE, in which:

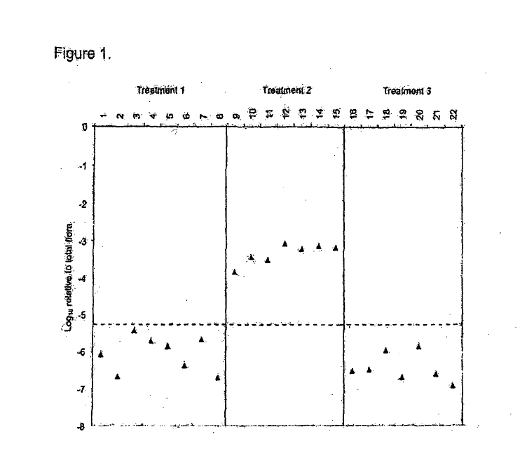

FIG. 1 shows a plot of colonisation of C. jejuni relative to the total flora in chicken caecum at day 28, measured with real-time PCR. There were not found any C. jejuni positive chickens within the negative control group (treatment 1) or in chickens with sorbate- and formic acid treatment (treatment 3). All chickens within the positive control group (treatment 2) were C. jejuni positive. Dashed line indicates the detection limit.

### EXAMPLES

### Example 1

### Materials and Methods

All in vivo experiments were started with 1-day-old conventional broiler chickens (Ross 308) of mixed sex. Chickens challenged with C. jejuni appeared healthy and showed no signs of disease. All in vivo experiments were approved by the Norwegian Research Authority.

### 1 Experimental Infections

### 1.1. Selection of Challenge Strain and Establishment of an Infection Model

Three C. jejuni strains were compared in vitro for sensitivity for benzoic acid and sorbic acid at two different pH values. These strains were C. jejuni strain 484 (from poultry leg), C. jejuni strain 523 (from poultry faeces), and C. jejuni strain 534 (from poultry faeces). Different combinations of pH (6.0 and 6.5) and concentrations of the acids (0.1, 0.01, 0.001, and 0.0001%) were tested in Mueller-Hinton broth (MH) with supplement for Campylobacter (SR232E; Oxoid Ltd., Basingstoke, UK). The strains were incubated in microaerophilic atmosphere for 48 hours at 42° C., and growth was measured by spectrophotometer.

The same three C. jejuni strains (strain 484, 523, and 534) were later examined for their colonisation ability in chicken in vivo. Three birds were assigned at random to each of three cages, in total 9 chickens. On day 14 two birds per cage were inoculated with the respective strain (as described in section 1.4). On day 17 all birds were put down (euthanised) and their caecal contents were examined quantitatively for C. jejuni counts.

A new experiment using C. jejuni strain 484 was conducted to obtain the optimum challenge dose. Each treatment group consisted of two cages with 4 chickens per cage, in total 32 chickens. Two different challenge doses were tested, high-dose (challenge dose of 10,000 colony forming units (cfu) per bird) and low-dose (challenge dose of 100 cfu per bird). Group A with non-inoculated cages was located on the wall separate from inoculated cages, whereas group B with non-inoculated cages was located next to inoculated cages: group C with low-dose inoculated cages and group D with high-dose inoculated cages. The broilers were challenged at 14 days of age as described in section 1.4.

Commercial broiler feeds supplemented with 70 ppm narasin were used throughout. The birds were offered a starter feed up to day 10, and a grower feed during the last part of the experiment. Environmental samples from the experimental room and swabs from chickens collected on day 0 and 13 were examined for C. jejuni with negative result. In order to test for risk of cross-contamination, non-inoculated cages were located in the same room as inoculated cages. To test if distance between cages is of importance, two non-inoculated cages were located on a separate wall, whereas the two other non-inoculated cages were located next to inoculated cages. Non-inoculated cages were sampled and examined for C. jejuni simultaneously with inoculated cages.

The cloacal mucosa of all birds in each cage was swabbed on days 1, 3, 8 and 15 after inoculation. A total of 63 cloacal swabs were collected from birds assigned to groups C and D (inoculated birds). Thirty-one of these were examined qualitatively and 32 were examined quantitatively. Cloacal swabs were put into separate tubes with 1.5 ml of buffered peptone water (BPW) and transported to the laboratory where analysis was commenced immediately.

1.2. Effect of In-Feed Formic Acid and Sorbate on C. jejuni Colonisation

The experimental groups differed with regard to challenge and feed additives. There was one basic recipe for the feed used in this study. The feed was equivalent to commercial pelleted grower feeds for broilers (wheat, soybean meal and oats were main ingredients), and it contained 70 ppm narasin. Treatment 1 was negative controls, with no. C. jejuni-challenge and acid-free feed. Treatment 2 was positive controls, with C. jejuni-challenge and acid-free feed. Treatment 3 were challenged with C. jejuni on day 13 and offered feed supplemented with 1% formic acid and 0.1% potassium sorbate from day 0.

All birds were examined for C. jejuni on day 0 and on day 13 (immediately before challenge), with negative results. The birds were housed in six separate cages. There were three experimental groups, each consisting of two separate cages. Each cage contained 4 one-day-old chickens, in total 24 chickens. The experimental groups (treatment 2 and 3) were inoculated with a suspension of C. jejuni strain 484 at day 13 as described in section 1.4. A total of eight faecal samples (2-4 fresh caecal 0.10 droppings and 4-6 cloacal swabs) per treatment group (one sample per bird) were collected on each of days 1, 3 and 8 after challenge. The experiment ended on day 28 (day 15 post-challenge), when caecal contents were Collected from all chickens. Crop material (contents as well as mucosal tissue) was collected from five chickens from each experimental group.

1.3. Survival of C. jejuni in Crop

An experiment was performed with five broiler chickens. These chickens were challenged at day 14 with C. jejuni strain 484 (as described in section 1.4). Samples from the crop and the caecae were examined for C. jejuni at day 25. Samples from the mucosal membranes and from luminal contents of both organs were examined separately.

Following collection of luminal crop contents for analyses based on real-time PCR and cultivation, the entire crop was removed from the carcass and divided into two equally sized parts. The mucosal membranes were flushed with sterile physiological saline for removal of luminal material before the surface was scraped off with a sterile scalpel blade. The mucosal scraping and the wall from each crop half were pooled as one sample and transported to the laboratory for C. jejuni analysis. One half was examined by real-time PCR, and the other half of the crop was examined by cultivation for C. jejuni.

Caecal contents from one caecum were examined by cultivation and contents from the other caecum were examined by real-time PCR. The caecal mucosa was flushed with sterile physiological saline and rubbed gently with a sterile surgical glove finger, in order to remove luminal contents. Mucosal scrapings from the narrow part of one caecum and the wide part of the other caecum were pooled as one sample and a corresponding pooled sample was collected from the opposite parts of the same caecae. From each bird one of these caecal samples was examined by cultivation, whereas the other sample was examined using real-time PCR.

### 1.4. Bacterial Challenge Procedure

C. jejuni strain 484 was used in the experiments. In order to make the inoculum, a single colony was inoculated into 10 ml BPW and incubated at 37±1° C. for 24 hours. The culture was serially diluted in BPW and the appropriate dilutions were used for inoculation of the chickens in the experiments.

The chickens were inoculated individually by crop instillation with approximately 1.5 ml of the bacterial suspension (depending on the concentration of the inoculum and the infection dose), using a 2 ml syringe with an attached flexible tube. The negative controls were inoculated with sterile BPW.

2 Examinations for C. jejuni

### 2.1. Sample Types and Examination

Cloacal swabs were used for pre-inoculation control of birds (qualitative cultivation) and for post-inoculation qualitative and quantitative cultivation. Swabs were also used for pre-inoculation control of the experimental premises (qualitative cultivation), for post-inoculation qualitative and quantitative cultivation from small intestinal and caecal dropping as well as contents, and for quantitative PCR-based examinations (real-time PCR) of caecal droppings and contents. Luminal contents of crop and caecae were used for post-inoculation quantitative cultivation and PCR-based examinations. Luminal contents of small intestine were also used for post-inoculation quantitative cultivation. Specimens of crop mucosa and caecal mucosa were used for post-inoculation quantitative cultivation and PCR-based examinations.

### 2.2. Cultivation Procedures

For detection of C. jejuni, each swab sample was immersed in 1.5 ml BPW in a test tube and transported to the laboratory for immediate analysis. The test tubes were shaken briefly on a whirl mixer and a loopful of broth was plated on modified charcoal cephoperazone desoxycholate agar (mCCDA). The plates were incubated in anaerobic jars under microaerobic conditions at 41.5±1.0° C. for 44±4 hours. A total of five typical colonies, with a flat or convex greyish and glossy surface, from each presumptive positive sample were sub-cultured on blood agar plates (BA) and incubated at 37±1° C. for 44±4 hours. Colonies with a typical colony morphology and typical appearance by light microscopy and which were positive in the catalase and oxidase tests, were further subjected to a hippurat test. Colonies that were positive on the hippurat test were defined as Campylobacter spp. Presumptive Campylobacter spp. were further identified to species level by using a multiplex-ID PCR (Johannessen et al. 2007 Lett Appl Microbiol 44: 92-97).

In order to enumerate Campylobacter from cloacal swabs, the swabs were moistened in BPW and weighed before taking the faecal sample. After collection of faeces the swab was weighed again and put in 1.5 ml of BPW. Samples were mixed with a whirl mixer before making a tenfold dilution series. Aliquots of 100 from the appropriate dilutions were spread on mCCDA and incubated as described above. Colonies with a typical morphology were counted and confirmed as described previously. The number of Campylobacter present in 1 g faeces was subsequently calculated.

Samples of luminal contents from crop and caeca were initially diluted 1:10 with BPW and further serially diluted in BPW. Samples of crop mucosa and caecal mucosa were immersed in 1.5 ml BPW in a test tube and shaken with a whirl mixer before initial dilution 1:10 with BPW and further serial dilution in BPW. The samples were then processed for enumeration as described above. The detection limit was 100 cfu per gram (cfu/g).

### 2.3. DNA Isolation and Quantitative Real-Time PCR

Swabs with caecal lumen and crop lumen contents were separately mixed with 1 ml of Solution 1 (50 mM glucose, 25 mM Tris-HC1 pH 8.0, 10 mM EDTA pH 8.0). DNA isolation and purification was further performed using an automated procedure with silica particles (Bioclone Inc., San Diego, Calif.) as described earlier by Skånseng et al. (2006 Mol Cell Probes 20: 269-279). For crop samples from the study of the effect of in-feed formic acid and sorbate, 200 μl of the crop fluid was diluted 1:4 in 4 M guanidinium thiocyanate (GTC), and further treated as the caecum samples. For the detection of C. jejuni in the mucous membrane of crop and caecum, a part of the mucous membrane were transferred to a FastPrep® tube (Qbiogene Inc., Carlsbad, Calif.) containing 250 mg glass beads (106 microns and finer, Sigma-Aldrich, Steinheim, Germany) and 500 μl 4 M GTC. The samples were homogenised for 40 seconds in FastPrep instrument (Qbiogene), and further treated as the lumen samples.

Quantification of C. jejuni was performed relative to the total flora (as described by Skånseng supra). Universal 16S rDNA primers and probe (Nadkarni et al. 2002 Microbiology (UK) 148: 257-266) was used for quantification of the total flora. C. jejuni-specific real-time PCR was performed using the primer- and probe set described by Nogva et al. (2000 Appl Environ Microbiol 66: 4029-4036). The real-time PCR reaction was performed as earlier described by Skånseng et al. (2007 PLoS Pathog 3: e175).

### Results

### 1. Infection Model

Three C. jejuni strains were tested for in vitro growth at different combinations of pH (6.0 and 6.5) and concentrations of sorbic- and benzoic acid (0.1, 0.01, 0.001, and 0.0001%). The effects of the acids were best at pH 6.0, and sorbic acid had the highest growth reduction for all three C. jejuni strains.

All three C. jejuni strains were tested in vivo, to examine their ability to colonise chicken caecum. C. jejuni strain 484 showed substantially higher caecal counts than strain 523, and also higher mean counts than strain 534. C. jejuni strain 484 was therefore chosen for further investigation. Two different inocula, high and low challenge dose (10,000 cfu and 100 cfu per chicken, respectively), were tested. The results showed that at 15 days post-inoculation, only 4/8 of the chickens with low-challenge dose were infected while 8/8 of the chickens in the high-challenge dose were infected with C. jejuni already on day 8 post-inoculation. The high-challenge dose was therefore chosen for the main experiment investigating the colonisation of both the caecum and the crop.

The main experiment was performed using bacteriocidal feed additives (sorbate and formic acid) for modifying the crop environment. From day 21 (day 8 post-inoculation) all chickens tested with treatment 2 (positive control) were positive for C. jejuni, while chickens with treatment 1 (negative control) and 3 (sorbate and formic acid) were negative for C. jejuni throughout the whole period. An analysis of variance (ANOVA) was performed on the real-time. PCR colonisation data, and there was a significant difference between the different treatments at day 21 (p-value<0.012). At day 28 the C. jejuni positive chickens had a colonisation level of approximately −3 log₁₀ relative to the total flora (FIG. 1), which corresponded to a cultivation-based level of log, 8.0-8.5 C. jejuni per gram caecal contents. The cultivation based results showed similar colonisation pattern as the quantification using real-time PCR.

2 Spatial Distribution of C. jejuni in Chicken Crop and Caecum

Real-time PCR and cultivation examinations were used on mucosa and lumen contents to determine the main location of C. jejuni (section 1.3). Based on real-time PCR we found that the mucosal C. jejuni counts were higher than luminal counts in all 5 chickens examined, and the mucosa in crop contained significantly higher (p<0.006, using ANOVA) level of C. jejuni relative to the total flora than the lumen contents. The relative level of C. jejuni in the mucosa of caecum was slightly higher than in lumen contents, but not significantly. The mucosa in crop contained about 2 log values more C. jejuni relative to the total flora than the lumen contents, and for caecum the difference was about 1 log value between the mucosa and the lumen contents.

TABLE 1
Colonisation of C. jejuni in mucosa and luminal contents of
caecum and crop, measured by real-time PCR. The amount of
C. jejuni is given in log₁₀ relative to the total flora.

| Sample | Caecum | Crop |
|---|---|---|
| Mucosa | | |
| 1 | −3.06 | −2.62 |
| 2 | −2.92 | −2.23 |
| 3 | −1.37 | −2.62 |
| 4 | −3.46 | −1.98 |
| 5 | −1.91 | −4.21 |
| Lumen | | |
| 1 | −4.07 | −4.76 |
| 2 | −4.23 | −4.25 |
| 3 | −2.43 | −3.64 |
| 4 | −3.34 | −4.63 |
| 5 | −3.34 | −5.34 |

We found that the relative level of C. jejuni in crop was significantly higher in the mucosa than in the lumen contents, an observation which is in accordance with a mucosal colonisation of the crop by C. jejuni. A mucosal colonisation strongly suggests that the crop is a reservoir for C. jejuni. A likely mechanism of chicken colonisation is that C. jejuni colonise the crop before subsequent colonisation of the lower part of the GI tract.

### Example 2

Following the experimental methodologies described in Example 1 (1.2), with the exception that Campylobacter challenge occurred at day 15, a further two formic acid and sorbate dosage regimes were tested. In these experiments treatment of the birds with 1.5% formic acid and 0.1% potassium sorbate from either day 0 or day 10 caused a reduction in the colonisation of the crop and caecum with Campylobacter at day 28. Treatment with 2% formic acid and 0.1% potassium sorbate from either day 0 or day 10 prevented colonisation of the crop and caecum with Campylobacter at day 28. Some evidence was obtained to suggest that treatments beginning at day 10 were slightly more effective than treatments beginning at day 0.

### Example 3

Chickens were reared in 12 pens of 100 birds. Chickens given feed with 1.5% formic acid and 0.1% potassium sorbate from 0 or 10 days of age displayed no substantially negative or positive effect on accumulated growth and feed efficiency measured at 28 days of age compared to control animals receiving equivalent feed without acid additive. Some evidence was obtained to suggest that birds receiving treatment beginning at day 10 performed slightly better than those receiving treatment beginning at day 0.

1. A method of treating a bird a Campylobacter species present in the crop of a bird, comprising
exposing the bird is exposed to an antimicrobial agent one or more times, and wherein least one exposure takes place in the first 20 days of the bird's life.

2. The method of claim 1, wherein said Campylobacter species is in a colony in the crop.

3. The method of claim 1, wherein said antimicrobial agent or a composition in which it is contained target the crop of a bird.

4. The method of claim 3, wherein the antimicrobial agent or composition in which it is contained is mucoadhesive.

5. The method of claim 1, wherein the antimicrobial agent or composition in which it is contained selectively releases and/or is activated in the crop.

6. The method of claim 1, wherein exposing is administering with or as part of the feed and/or drinking water of the bird.

7. The method of claim 6, further comprising exposing the bird to an in-feed antimicrobial after a period of feed withdrawal.

8. The method of claim 7, wherein said period of feed withdrawal is sufficient to stimulate the appetite of the bird to a degree sufficient to cause the bird to accumulate the feed and the antimicrobial in the crop of the bird.

9. The method of claim 1, wherein the antimicrobial agent is co-administered with an agent to activate or enhance the action of the antimicrobial agent in the crop.

10. The method of claim 1, wherein the antimicrobial agent is an organic acid or a salt thereof.

11. The method of claim 10, wherein the organic acid is benzoic, sorbic or formic acid.

12. The method of claim 10, wherein the salt of the organic acid is the potassium, magnesium or calcium salt.

13. The method of claim 10, wherein the organic acid or salt thereof is co-administered with a proton donor.

14. The method of claim 1, wherein the antimicrobial agent is a benzaldehyde or an azole.

15. The method of claim 1, wherein the antimicrobial agent is trisodium phosphate.

16. The method of claim 1, wherein the antimicrobial agent is at least one of cowberry, rowanberry, blueberry or leek or an extract therefrom.

17. The method of claim 16, wherein the antimicrobial agent is co-administered with at least one degrative enzyme, selected from a pectinase, a cellulase and a protease.

18. The method of claim 1, wherein the antimicrobial agent is an antimicrobial peptide.

19. The method of claim 18, wherein the antimicrobial agent is co-administered with at least one peptidase.

20. A product containing (a) an antimicrobial agent, and (b) an activator and/or enhancer of said antimicrobial agent, as a combined preparation for separate, simultaneous or sequential use in the treatment of a Campylobacter species in the crop of a bird, wherein the bird is exposed to said antimicrobial agent one or more times, wherein at least one exposure takes place in the first 20 days of the bird's life.

21. (canceled)

22. The method of claim 2, wherein said Campylobacter species is in a colony in the mucosa of the crop.

23. The method of claim 3, wherein said antimicrobial agent or composition in which it is contained targets the mucosa of the crop.
